(19) United States
(12) Patent Application Publication
(10) Pub. No.: US 2009/0036650 A1
(43) Pub. Date: Feb. 5, 2009

(54) Compounds and methods for peptide synthesis

(76) Inventors: Julio Herman Cuervo, Arlington, MA (US); Milka Yanachkova, Shrewsbury, MA (US); Russell C. Petter, Stow, MA (US); Thomas F. Durand-Reville, Cambridge, MA (US); Jose Carlos Jimenez-Garcia, Brookline, MA (US)

Correspondence Address: STEPTOE & JOHNSON LLP, 1330 CONNECTICUT AVENUE, N.W., WASHINGTON, DC, 20036, US

(21) Appl. No.: 11/886,875

(22) PCT Filed: Mar. 24, 2006
(86) PCT No.: PCT/US2006/010872

§ 371 Date: Apr. 30, 2008

Related U.S. Application Data
(60) Provisional application No. 60/664,945, filed on Mar. 25, 2005.

(51) Int. Cl. C07K 1/04; C07K 2/00

(52) U.S. Cl. 530/334; 530/300

(57) ABSTRACT

A backbone nitrogen modifying group can prevent aggregation of peptides during peptide synthesis. The modifying group can promote aqueous solubility of the peptides, and be compatible with solid phase peptide synthesis. Methods for making peptides are also described.

### CLAIM OF PRIORITY

This application claims priority under 35 USC § 119(e) to U.S. Patent Application Ser. No. 60/664,945 filed on Mar. 28, 2005, which is incorporated by reference in its entirety.

### TECHNICAL FIELD

This invention relates to compounds and methods for peptide synthesis.

### BACKGROUND

Solid phase peptide synthesis (SPPS) can be used to prepare a wide variety of peptide sequences. Some sequences, however, can be difficult to prepare by standard methods of SPPS. These difficult sequences are often characterized by a high content of hydrophobic amino acids and a tendency to adopt a β-sheet secondary structure. Peptides that have a difficult sequence can aggregate during synthesis. Aggregation can have a negative impact on the yield, purity, and water solubility of the final peptide product.

### SUMMARY

Protecting the backbone nitrogen of a peptide can prevent aggregation during synthesis. The backbone nitrogen can be modified by a group that interferes with the formation of hydrogen bonds involving the backbone nitrogen, which in turn can interfere with the formation of a β-sheet structure. β-sheet structures can sometimes lead to aggregation of peptides during synthesis. In particular, including a backbone nitrogen modifying group can prevent or reduce the occurrence peptide aggregation during Fmoc- or Boc-based solid phase peptide synthesis. The modifying group can disrupt formation of hydrogen bonds involving the backbone nitrogen. The modifying group can enhance the aqueous solubility of a peptide, and facilitate purification and characterization of the peptide. The modifying group can be compatible with reactions conditions used in Fmoc and Boc synthesis, and does not interfere with chemical peptide ligation. Desirably, the modifying group can be easily removed from the final peptide product.

In one aspect, a method of making a peptide includes forming a peptide including a backbone nitrogen modifying group which includes a substituted aryl group. The substituted aryl group includes a directing moiety and a hydrophilic moiety.

The peptide can be linked to a solid support. The peptide can include at least one commonly occurring natural amino acid residue which optionally includes a protecting group. The peptide can include at least one non-naturally occurring amino acid residue.

The method can include adding an amino acid residue to the peptide, thereby extending the peptide. The method can include cleaving the peptide from the solid support without substantially removing the backbone nitrogen modifying group from the peptide. The method can include removing the backbone nitrogen modifying group from the peptide.

The substituted aryl group can be a substituted phenyl group. The substituted phenyl group can be ortho-unsubstituted (i.e., unsubstituted in the 2- and 6-positions). The hydrophilic moiety can include a tertiary amine. The peptide can be substantially water-soluble.

The peptide can have the formula:

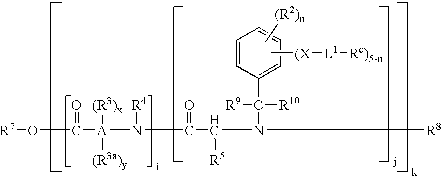

X is O, S, NH, or a bond. Each L¹, independently, is C₁-C₁₀ alkylene, alkenylene, alkynylene, cycloalkylene, arylene, or aralkylene, where L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—. Each R^{c}, independently is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl.

Each R², independently, is hydrogen, —R^{a}, —OR^{a}, —SR^{a}, —NR^{a}R^{b}, —NR^{a}C(═O)R^{b}, or halo. R² can be optionally substituted with -L¹-R^{c}. Each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl. Each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl.

Each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl. Each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl.

Each R⁹, independently, is hydrogen, alkyl, aryl, substituted alkyl, substituted aryl, or halo; and each R¹⁰, independently, is hydrogen, alkyl, aryl, substituted alkyl, substituted aryl, or halo.

Each A, independently, is C₁-C₁₀ alkylene, alkenylene, alkynylene, cycloalkylene, arylene, or aralkylene. A optionally includes 1-3 heteroatoms selected from N, O and S.

Each R³, independently, is hydrogen, alkyl, alkenyl, alknyl, cycloalkyl, heterocycloalkyl, aralkyl, (cycloalkyl)alkyl, (heterocycloalkyl)alkyl or aryl. R³ is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}.

Each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl. R^{3a} is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}. R³ and R^{3a} together with the atoms to which they are attached can form a 3-14 membered cyclic, bicyclic or tricyclic moiety, which optionally includes 1-6 heteroatoms selected from N, O, and S.

Each R⁴, independently, is hydrogen or alkyl. R³ and R⁴ together with the atoms to which they are attached form a 3-14 membered cyclic, bicyclic or tricyclic moiety, which optionally includes 1-6 heteroatoms selected from N, O, and S.

Each R⁵, independently, is hydrogen, alkyl, alkenyl, alkynyl, cycloalkyl, heterocycloalkyl, aralkyl, (cycloalkyl)alkyl, (heterocycloalkyl)alkyl or aryl. R⁵ is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}.

Each R^{f} and each R^{g}, independently, is hydrogen, alkyl, aryl, aralkyl, or a protecting group.

R⁷ is hydrogen, alkyl, aryl, aralkyl, or a solid support. R⁸ is hydrogen, alkyl, an amino protecting group, or has the formula —C(═O)CH(R⁵)R⁶, where R⁶ is a leaving group.

Each i, and each j, independently, is zero or a positive integer. k is a positive integer, m is 1 or 2, n is 0, 1, 2, 3 or 4. Each x, independently, is 1, 2, 3, 4, or 5, each y, independently, is 1, 2, 3, 4, or 5, and z is 1, 2, 3, 4, 5 or 6.

In certain circumstances, the peptide includes at least one -L¹-R^{c}. The peptide can have the formula:

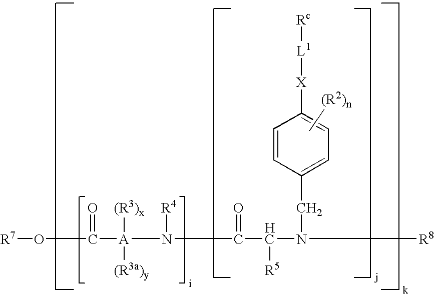

X can be O. L¹ can be C₁-C₄ alkylene. R^{c} can be heterocycloalkyl. In particular, —X-L¹-R^{c} can be 2-(morpholin-4-yl)ethoxy. Each R⁵, independently, can be hydrogen or alkyl. When R⁷ is a solid support, R⁸ can be an amino protecting group. Each A can be C₁ alkylene and each R^{3a} can be hydrogen. The total of all i and all j can be less than 300. The peptide can have a molecular weight of no greater than 40 kDa. R⁸ can have the formula —C(═O)CH(R)R⁶, where R⁶ is a leaving group. R⁶ can be halo. When used in the method, each j can be zero.

The method can include contacting the peptide with a compound having the formula:

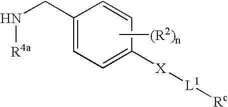

where X, L¹, R^{c}, R² and n are defined above. In the compound, R^{4a} can be hydrogen, alkyl, or an amino protecting group. The compound can be 4-(2-(morpholin-4-yl)ethoxy)benzylamine. The method can include adding an amino acid residue to the peptide, thereby forming a longer peptide.

The method can include contacting the peptide with a compound having the formula:

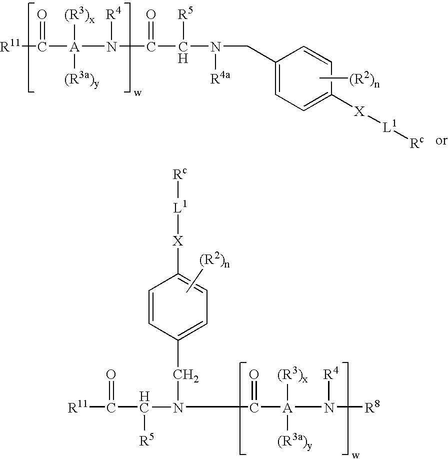

where X, L¹, R^{c}, R², R^{3a}, R⁴, R⁵, x, y, and n are defined above. In the compound, R^{4a} is hydrogen, alkyl, or an amino protecting group. R⁵ is hydrogen, alkyl, alkenyl, alkynyl, cycloalkyl, heterocycloalkyl, aralkyl, (cycloalkyl)alkyl, heterocycloalkyl)alkyl or aryl. R⁵ is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}. R¹¹ is hydroxy, alkoxy, aryloxy, aralkyloxy, or a leaving group. w is 0, 1, or 2.

In another aspect, a composition includes a peptide including a backbone nitrogen modifying group including a substituted aryl group, which includes a directing moiety and a hydrophilic moiety. The peptide can include a plurality of backbone nitrogen modifying groups.

The peptide can have the formula:

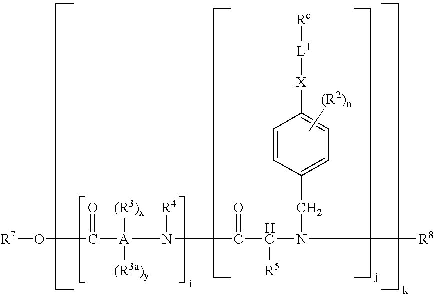

as described above.

In another aspect, a compound having the formula:

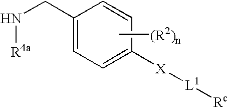

where X, L¹, R^{c}, R² and n are defined above. In the compound, R^{4a} can be hydrogen, alkyl, or an amino protecting group.

In another aspect, a compound having the formula:

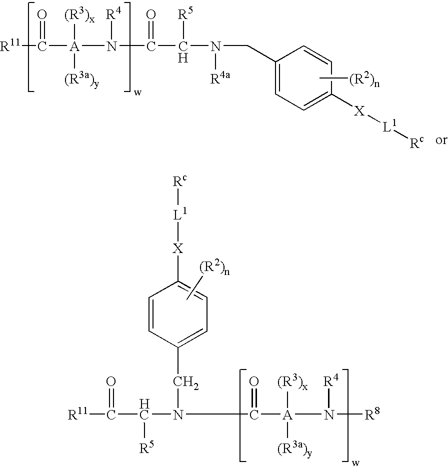

where X, L¹, R^{c}, R², R⁴, and n are defined above. In the compound, R^{4a} is hydrogen, alkyl, or an amino protecting group. R⁵ is hydrogen, alkyl, alkenyl, alkynyl, cycloalkyl, heterocycloalkyl, aralkyl, (cycloalkyl)alkyl, (heterocycloalkyl)alkyl or aryl. R⁵ is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}. R¹¹ is hydroxy, alkoxy, aryloxy, aralkyloxy, or a leaving group. w is 0, 1, or 2.

In yet another aspect, a method of making a peptide having a predetermined amino acid sequence includes determining a beta-sheet-forming propensity for at least a portion of the amino acid sequence, and selecting an amino acid residue of the sequence for modification with a backbone nitrogen modifying group based on the determined beta-sheet-forming propensity. The backbone nitrogen modifying group can include a substituted aryl group which includes a directing moiety and a hydrophilic moiety.

The details of one or more embodiments are set forth in the description below. Other features, objects, and advantages will be apparent from the description, and from the claims.

### DETAILED DESCRIPTION

The formation of β-sheet structure and concomitant peptide aggregation remains one of the most difficult challenges to overcome during solid phase peptide synthesis of peptides that contain regions capable of forming β-sheet structure or aggregating. Most of the common available methods help to prevent aggregation in the early steps of SPPS, but these methods lack effectiveness in late steps in the synthesis of the peptide (e.g., during purification and characterization). In the late steps, the peptide is removed from the solid support and deprotected, and instead of a pure, soluble product, an insoluble crude peptide which can be difficult to purify and characterize is obtained. Current methods to prevent aggregation can also be incompatible with standard SPPS protocols.

A β sheet is made of several β-strands arranged side-by-side. The peptide bonds in a β-strand adopt an almost fully extended conformation. The side chains of adjacent amino acids in a β-strand point in opposite directions. A β-sheet is formed by linking two or more β strands by hydrogen bonds. Adjacent chains in a β-sheet can run in opposite directions (an antiparallel β-sheet) or in the same direction (a parallel β-sheet). In the antiparallel arrangement, the NH group and the CO group of each amino acid are respectively hydrogen bonded to the CO group and the NH group of a partner on the adjacent chain. In the parallel arrangement, the hydrogen-bonding scheme is slightly more complicated. For each amino acid, the NH group is hydrogen bonded to the CO group of one amino acid on the adjacent strand, whereas the CO group is hydrogen bonded to the NH group on the amino acid two residues farther along the chain. Many strands, typically 4 or 5 but as many as 10 or more, can come together in β-sheets. Such β-sheets can be purely antiparallel, purely parallel, or mixed.

A region of a peptide is prone to forming β-sheet structure if it is known or believed to form a β-sheet structure. For example, hydrophobic residues are often found in beta-sheet structures. The propensity of a particular region to form β-sheet structure can be predicted or estimated (see, for example, Smith, C. K., et al., Biochemistry 1994, 33(18):5510-7; and Creighton, T. E. Proteins, 2^{nd} ed., W.H. Freeman and Co., New York., 1993, each of which is incorporated by reference in its entirety).

When a synthetic peptide forms a β-sheet structure during synthesis, it can cause aggregation of the peptides, leading to undesirable results such as poor yield or insoluble products. Because β-sheets rely on hydrogen bonding between backbone nitrogen and carbonyl oxygen atoms, the formation of β-sheets can be prevented by blocking hydrogen bonding at these positions. A backbone nitrogen protecting group can block hydrogen bonding. Preferably, the protecting group is compatible with common SPPS conditions, contributes to aqueous solubility of the peptide, and is selectively removable when the synthesis is complete. Selection of residues to be protected can be guided by a known secondary structure (e.g., as revealed by X-ray or NMR structural determination), an inferred secondary structure (e.g, based on sequence homology), or a predicted secondary structure. A prediction or estimation of β-sheet forming propensity can guide the selection of residues to include a backbone nitrogen protecting group. In particular, the backbone nitrogen of one or more residues in regions prone to forming P-sheet structures can be protected.

Solid phase peptide synthesis is described in, for example, Weng C. Chan, and Peter D. White: Fmoc Solid Phase Peptide Synthesis: A Practical Approach, 2000, Oxford University Press; and John Jones, Amino Acid and Peptide Synthesis, 2002, Oxford University Press, each of which is incorporated by reference in its entirety. Solid supports used in solid phase peptide synthesis can include, for example, a Merrifield resin, a Wang resin, or a Rink resin.

In general, a peptide is a compound including a peptide bond:

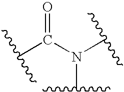

Typically, the peptide is formed by condensation of an amine with a carboxylic acid. The peptide can be a polypeptide, in other words, including two or more peptide bonds. The peptide can have a backbone (which includes the peptide bonds) and one or more side chains. In general the side chain is a substituent that branches from the backbone.

A peptide can be formed by the condensation of amino acids. An amino acid is a compound including an amino group and a carboxylic acid group. The amino acid can also include a side chain. For example, an amino acid can have the formula:

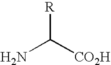

where R represents the side chain. In general the side chain of an amino acid is a substituent that branches from a backbone connecting the amino group to the carboxylic acid group. The amino acid can be an alpha amino acid (as shown above, where the amino group is attached to the position alpha to the carboxylic acid group), a beta amino acid, a gamma amino acid, etc. When a peptide is formed by the condensation of amino acids, the peptide can be described as including an amino acid residue. An amino acid residue refers to the portion of the peptide derived from a particular amino acid. For example, the residue that results when the amino acid alanine,

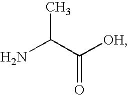

is incorporated into a peptide is

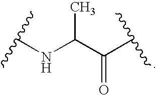

A polypeptide can be formed by sequential condensation of several amino acids.

A peptide can include any of the commonly naturally occurring amino acid residues (Ala, Cys, Asp, Glu, Phe, Gly, His, Ile, Lys, Leu, Met, Asn, Pro, Gln, Arg, Ser, Thr, Val, Trp, and Tyr) in either stereochemical form (i.e., in D- or L-form). A peptide can include other amino acid residues, such as a modified form of a common naturally occurring amino acid, or an unnatural amino acid. A wide variety of non-naturally occurring amino acids are commercially available for use in SPPS, for example from Novabiochem, Sigma-Aldrich and other vendors.

It can be desirable to use protecting groups during SPPS. The protecting groups are linked to potentially reactive sites and prevent undesired reactions from occurring. Preferably, a protecting group can be removed selectively and completely. In SPPS, protecting groups can be used for potentially reactive amino acid side chains, such as the side chains of Ser, Thr, Trp, Arg, Lys, Cys, His, Asp, Glu, Tyr, etc., and at the N- or C-terminus of the peptide. The protecting groups can include, for example, tBoc, Fmoc, Cbz, Bz, etc. See, for example, Theodora W. Greene, Peter G. M. Wuts: Protective Groups in Organic Synthesis, 3^{rd} ed. Wiley Interscience, 1999, which is incorporated by reference in its entirety. For example, a hydroxyl group (as in the side chains of Ser and Thr) can be protected with, for example, a t-butyl group or a benzyl group. Amino groups can be protected with, for example, Boc or Fmoc protecting groups. Other protecting groups for reactive side chains are known.

The use of a backbone nitrogen protecting group can facilitate synthesis of difficult sequences. See, for example, Johnson, T., et al., J. Chem. Soc. Chem. Commun. 1993, 369-372; White, P., et al., J. Peptide Sci. 2004, 10, 18-26; and Johnson, T. and Quibell, M., Tetrahedron Lett. 1994, 35, 463-466, each which is incorporated by reference in its entirety.

Backbone nitrogen protecting groups such as methyl, benzyl, and p-methylbenzyl can block β-sheet formation during routine solid phase peptide synthesis. This, in turn can block the aggregation induced by formation of hydrogen bonds, such as between β-strand structures. However, these backbone nitrogen protecting groups can be difficult to remove by hydrogenation or HF, and do not promote solubility of the resulting peptides in aqueous buffers.

When used as a backbone nitrogen protecting group, a 4-methoxy-substituted benzyl group can have higher acid lability can the corresponding 4-methyl-substituted benzyl group (see, for example, Johnson, T. and Quibell, M., Tetrahedron Lett. 1994, 35, 463-466). A 4-methoxy-substituted benzyl group can be removed by HF or hydrogenation. It does not, however, effectively promote the solubility of the protected peptide fragments in aqueous buffers.

A backbone nitrogen modifying group can prevent formation of hydrogen bonds involving the backbone nitrogen of a peptide. This in turn prevents or reduces the extent of β-sheet formation during SPPS. The modifying group preferably promotes the solubility of peptides in aqueous buffers and is compatible with standard SPPS protocols. The peptide including the modifying group can be substantially water soluble, in other words, soluble in water, an aqueous buffer, or a water-solvent mixture. The peptide can be soluble at concentrations of less than 10 mg/mL, less than 1 mg/mL, or less than 0.1 mg/mL.

In general, a backbone modifying group can include a substituted aryl group, such as, for example, a substituted phenyl group. The substituted aryl group can include a directing moiety and a hydrophilic moiety. A directing moiety is a substituent on an aromatic ring that affects the reactivity of the ring, such as by increasing or decreasing reactivity at one or more positions on the ring. For example, a hydroxy (—OH) substituent can be a para-directing moiety (i.e., influencing reactivity at the position para to the hydroxy substituent), and a nitro (—NO₂) substituent can be a meta-directing moiety (i.e., influencing reactivity at the position meta to the hydroxy substituent). The hydrophilic moiety can enhance the water solubility of a peptide including the modifying group. In particular, the hydrophilic moiety can the water solubility of a peptide compared to a peptide lacking the hydrophilic moiety on a backbone nitrogen modifying group. In certain embodiments, the directing moiety and the hydrophilic moiety can both belong to a single substituent on the aromatic ring. The aryl group can optionally be ortho-unsubstituted. For example, if the modifying group is a substituted benzyl group, the ortho-positions (i.e., the 2- and 6-positions) of the phenyl ring can be unsubstituted. The aryl group can be meta-substituted, para-substituted, or both meta- and para-substituted.

The modifying group can have the formula:

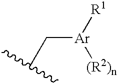

where Ar is an aryl group, R¹ includes a directing moiety and R² includes a hydrophilic moiety; or R¹ includes a both directing moiety and a hydrophilic moiety. The directing group can be, for example, an electron releasing group such as hydroxy, alkoxy, amino, alkylamino, or dialkylamino. The directing group can be in a para position. The hydrophilic moiety can include a heteroatom such as N, O, or S. The hydrophilic moiety can include a hydrophilic group such as hydroxy or a tertiary amine. The hydrophilic moiety can include a heterocyclic group. n can be 0, 1, 2, 3 or 4.

An aryl group is a cyclic aromatic group such as, for example, phenyl, naphthyl, indenyl, indanyl, azulenyl, fluorenyl, or anthracenyl; or a heterocyclic aromatic group such as, for example, furyl, pyridyl, pyrrolyl, oxazolyl, thiazolyl, imidazolyl, pyrazolyl, indolyl, benzo[b]furanyl, 2,3-dihydrobenzofuranyl, benzimidazolyl, purinyl, quinolinyl, isoquinolinyl, cinnolinyl, phthalazinyl, or quinazolinyl.

A heterocyclyl group is a cyclic group including one or more heteroatoms in the ring, typically N, O, or S. The heterocyclyl group can be monocyclic, bicyclic, tricyclic, or have four or more rings. When more than one ring is present, the rings can optionally be fused. The heterocyclyl group can be an aryl group, an unsaturated group (i.e., including one or more double bonds), or a saturated group (i.e., including only single bonds). A heterocycloalkyl group can be a saturated heterocylyl group. Some examples of heterocyclyl groups include tetrahydrofuryl, dihydrofuryl, furyl, oxazolyl, pyridyl, thioxazolyl, imidazolyl, benzimidazolyl, indolyl, pyrrolyl, pyrrolidinyl, morpholinyl, thiomorpholinyl, and dioxanyl.

A backbone nitrogen modifying group can have the formula:

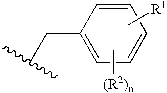

where:

R¹ is —R^{a}, —OR^{a}, —SR^{a}, —NR^{a}R^{b}, —NR^{a}C(═O)R^{b}, halo, or —X-L¹-R^{c}. Each R², independently, is hydrogen, —R^{a}, —OR^{a}, —SR^{a}, —NR^{a}R^{b}, —NR^{a}C(═O)R^{b}, or halo. R² is optionally substituted with -L¹-R^{c}.

Each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl. Each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl.

X is O, S, NH, or a bond. L¹ is C₁-C₁₀ alkylene, alkenylene, alkynylene, cycloalkylene, arylene, or aralkylene. L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—. R^{c} is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl. Each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl. Each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl. n is 0, 1, 2, 3 or 4, m is 1 or 2, and z is 1, 2, 3, 4, 5 or 6.

In some circumstances, the modifying group can have the formula:

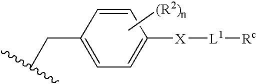

where R², X, L¹, R^{c} and n are defined above. The modifying group can be unsubstituted at the ortho-positions.

The modifying group can have the formula:

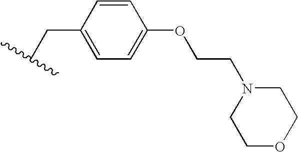

The modifying group can be incorporated into a peptide. The peptide can have the formula:

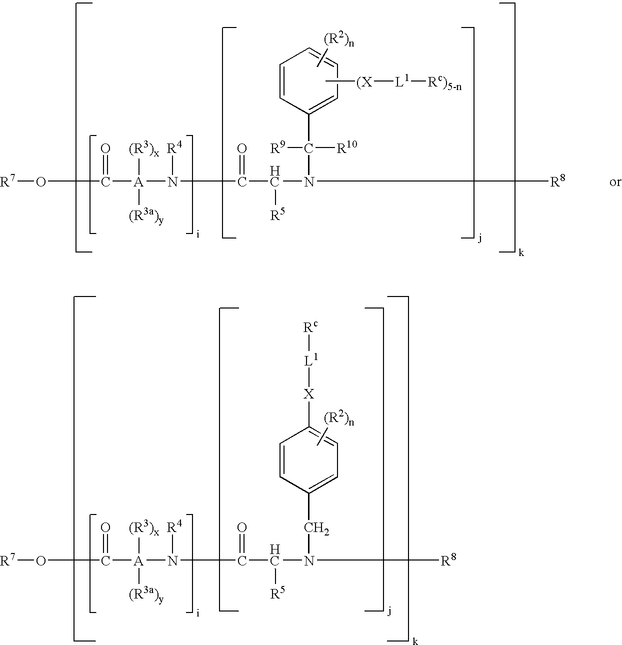

where R², X, L¹, R^{c}, R⁷, R⁹, and R¹⁰, are defined above. Each A, independently, is C₁-C₁₀ alkylene, alkenylene, alkynylene, cycloalkylene, arylene, or aralkylene. A optionally includes 1-3 heteroatoms selected from N, O and S. Each R³, independently, is hydrogen, alkyl, alkenyl, alkynyl, cycloalkyl, heterocycloalkyl, aralkyl, (cycloalkyl)alkyl, (heterocycloalkyl)alkyl or aryl. R³ is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}.

Each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl. R^{3a} is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}. R³ and R^{3a}, together with the atoms to which they are attached can form a 3-14 membered cyclic, bicyclic or tricyclic moiety, optionally including 1-6 heteroatoms selected from N, O, and S;

Each R⁴, independently, is hydrogen or alkyl. R³ and R⁴ together with the atoms to which they are attached can form a 3-14 membered cyclic, bicyclic or tricyclic moiety, optionally including 1-6 heteroatoms selected from N, O, and S.

Each R⁵, independently, is hydrogen, alkyl, alkenyl, alkynyl, cycloalkyl, heterocycloalkyl, aralkyl, (cycloalkyl)alkyl, (heterocycloalkyl)alkyl or aryl. R⁵ is optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}.

Each R^{f}, independently, is hydrogen, alkyl, aryl, aralkyl, or a protecting group. Each R^{g}, independently, is hydrogen, alkyl, aryl, aralkyl, or a protecting group.

R⁸ is hydrogen, alkyl, an amino protecting group, or has the formula —C(═O)CH(R⁵)R⁶, where R⁶ is a leaving group.

Each i, independently, is zero or a positive integer. Each j, independently, is zero or a positive integer. At least one j can be a positive integer. k is a positive integer; n is 0, 1, 2, 3 or 4; m is 1 or 2; each x, independently, is 1, 2, 3, 4, or 5; and each y, independently, is 1, 2, 3, 4, or 5.

In the peptide, X can be O, and L¹ can be C₁-C₄ alkylene. R_{c} can be —NR^{a}R^{b} or heterocycloalkyl. In particular, —X-L¹-R^{c} can be 2-(morpholin-4-yl)ethoxy. Each R² can be hydrogen. When A is C₁ alkylene, x and y can each be 1, and R^{3a} can be hydrogen. In some circumstances, each A is C₁ alkylene. When R⁷ is a solid support, R⁸ can be hydrogen or an amino protecting group. In the peptide, each i can be, for example, less than 50, less than 30, less than 20, less than 10 or less than 5. The sum of all i and j in the peptide can be, for example, less than 300, less than 200, less than 100, less than 75, less than 50, or less than 40. The molecular weight of the peptide (excluding R⁷, if R⁷ is a solid support) can be, for example, less than 40 kDa, less than 30 kDa, less than 20 kDa, less than 15 kDa, less than 10 kDa, or less than 5 kDa.

A method of making a peptide can include contacting a peptide having the formula:

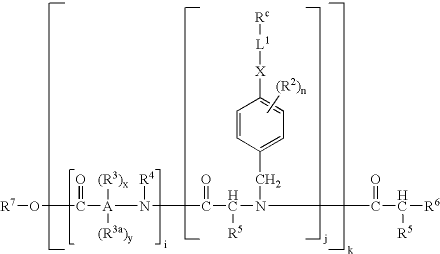

where R², R³, R^{3a}, R⁴, R⁵, X, L¹, R^{c}, R⁷, i, j, k, and n are defined above, and R⁶ is a leaving group;

with a compound of formula:

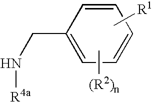

where R¹, R², R^{4a} and n are defined above.

In some circumstances the compound can have the formula:

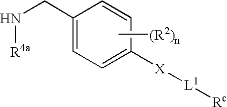

where X, L¹, R^{c}, R², R^{4a} and n are defined above.

The modifying group can be incorporated into an amino acid compound or an amino acid-derived compound. The compound can have the formula:

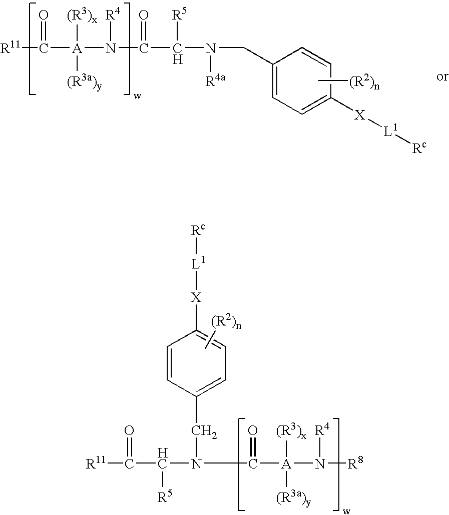

where X, L¹, R^{c}, R⁴, R⁵ and R² are defined above. Each R³ and each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl. R³ and R^{3a} are each independently optionally substituted with —OR^{f}, —SR^{f}, —CO₂R^{f}, halo, haloalkyl, —CN, —NO₂, —NR^{f}R^{g}, —C(═O)NR^{f}, —NHC(═NH)NR^{f}R^{g}; R⁴ is hydrogen, alkyl, or an amino protecting group. R³ and R⁴ together with the atoms to which they are attached can form a 3-14 membered cyclic, bicyclic or tricyclic moiety, optionally including 1-6 heteroatoms selected from N, O, and S. R^{4a} is hydrogen, alkyl, or an amino protecting group. R¹¹ is hydroxy, alkoxy, aryloxy, aralkyloxy, a solid support, or a leaving group. w is 0, 1, or 2. The amino acid or amino-acid derived compound can be used in solid phase peptide synthesis. When R¹¹ is a leaving group, R¹¹ can form an activated ester with carbonyl group to which it is attached. The activated ester can be, for example, a p-nitrophenyl ester, an N-hydroxysuccinimidyl ester, a pentafluorophenyl (OPfp) ester, a 3-hydroxy-2,3-dihydro-4-oxo-benzo-triazone (ODhbt) ester, a 1-hydroxybenzotriazole (HOBt) ester, or a 1-hydroxy-7-azabenzotriazole (HOAt) ester.

A para-methoxybenzyl group can be a backbone nitrogen modifying group. The para-methoxy substituent can act as a directing group, increasing the reactivity of the modifying group compared to analogous benzyl or para-methylbenzyl modifying groups. For example, para-methoxybenzyl can be more acid labile as a backbone nitrogen modifying group than benzyl or para-methylbenzyl. However, the para-methoxybenzyl group is a poor water-solubilizing moiety.

To increase water solubility without compromising acid lability of a 4-methoxybenzyl group, the methoxy group can be replaced with the more hydrophilic 2-(morpholin-4-yl)-ethoxy group. The backbone nitrogen modifying group 4-(2-morpholin-4-yl-ethoxy)benzyl (MEB) has been synthesized and used in solid phase peptide synthesis.

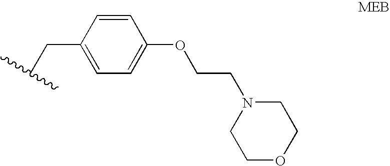

The backbone nitrogen modifying group can be installed on a growing peptide chain during solid phase synthesis. A protected amino acid can be added to a peptide chain by introducing an amino acid precursor to the growing chain. The amino acid precursor can include a carbonyl group, and an alpha carbon. The alpha carbon is linked to a side chain and to a leaving group. The leaving group can be displaced by an amino group of a compound including the modifying group, for example, in a nucleophilic substitution. In this way, a backbone nitrogen-protected amino acid is added to the growing peptide chain.

For example, the MEB modifying group can be installed on a peptide during solid phase synthesis. The free amino terminus of a solid-support bound peptide is allowed to react with an amino acid precursor, such as, for example, chloroacetyl chloride. The resulting chloroacetyl-substituted peptide can be reacted with MEBA, displacing the chloride leaving group. The amino nitrogen of MEBA becomes a backbone nitrogen of the peptide.

Scheme 1 illustrates the addition of a MEB-protected amino acid to a growing peptide chain. In Scheme 1, a polymer-bound peptide chain includes amino acid residues 1, 2, . . . , n−1 (with corresponding side chains indicated by R₁, R₂, . . . , Rₙ₋₁). The subsequent amino acid residue to be introduced (residue n), having a side chain indicated by Rₙ, is derived from a suitable precursor. For example, n can be less than 100, less than 75, less than 50, less than 40, less than 30, less than 20, or less than 10. The precursor can include an activated carbonyl group, and an α-carbon. The activated carbonyl group can be, for example, an acid halide such as an acid chloride or acid bromide, or an activated ester, such as, for example a succinimidyl ester, para-nitrophenyl ester, a pentafluorophenyl ester, or a 1-hydroxybenzotriazole ester. Other activated carbonyl group and other activated esters are known. In general, the activated carbonyl group includes a leaving group (shown as LG¹) linked to a carbonyl group. Rₙ, and a leaving group (shown as LG²) can be attached to the α-carbon. The leaving group LG² can be, for example, a halo group. For example, if residue n is glycine (Rₙ is hydrogen), the precursor can be chloroacetyl chloride or bromoacetic anhydride; if residue n is alanine (Rₙ is methyl), the precursor can be 2-chloropropionyl chloride. Once the precursor has been added to the growing peptide chain, the resulting peptide can be allowed to react with MEBA, thus adding the MEB-protected amino group of amino acid residue n. The synthesis of the peptide chain can then continue using standard procedures. As shown in Scheme 1, amino acid residue n+1 is added by reaction with the MEB-protected amino group of amino acid residue n.

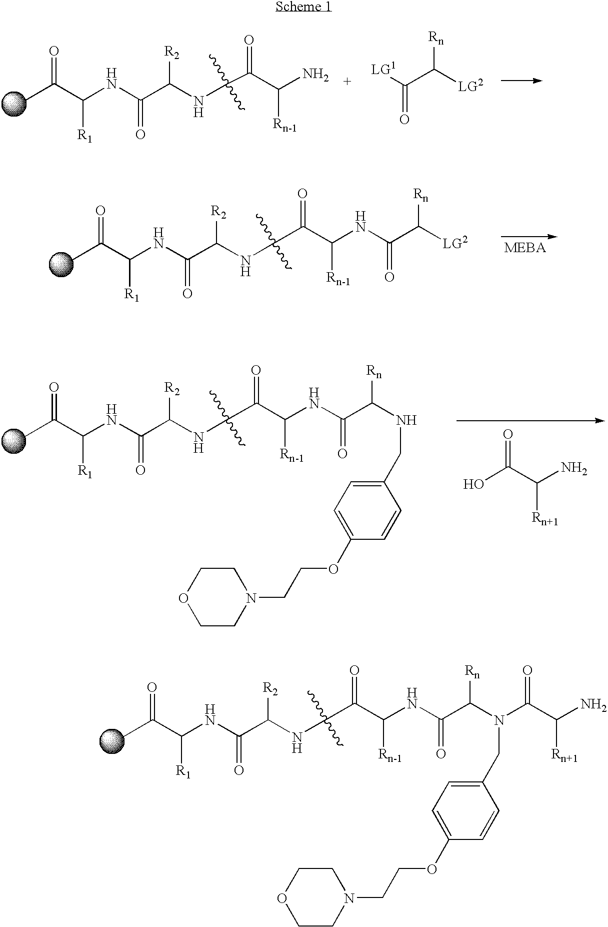

Rₙ can be, for example, hydrogen or alkyl. Rₙ can be H, —CH₃, —CH(CH₃)₂, —CH₂CH(CH₃)₂, or —CH(CH₃)CH₂CH₃, such that the amino acid residue at position n can be Gly, Ala, Val, Leu or Ile.

Alternatively, the MEB group can be first incorporated into an amino acid which is then added to a growing peptide chain during SPPS. As shown in Scheme 2, the carboxylic acid of a MEB-containing amino acid is coupled to the free amino group of a peptide linked to a solid support. The MEB-containing amino acid can include an amino protecting group, such as Fmoc (as shown in Scheme 2) or Boc. The MEB-containing amino acid can be deprotected to remove the amino protecting group, and a subsequent amino acid added to the peptide.

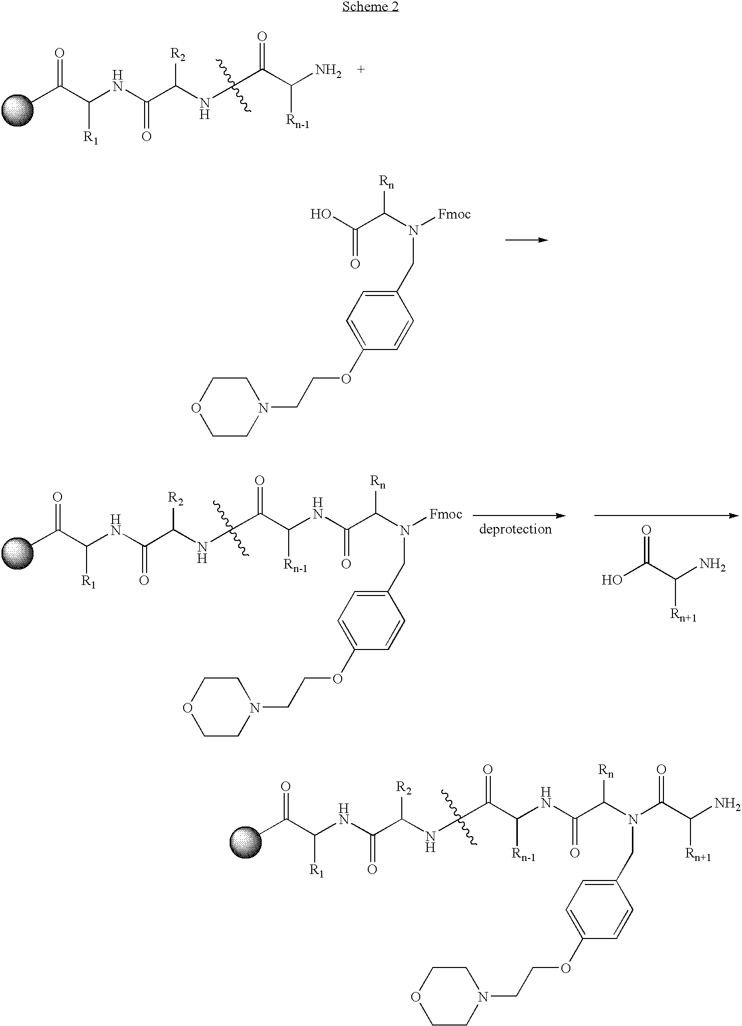

In another method, the MEB group can be first incorporated into a dipeptide which is then added to a growing peptide chain during SPPS. As shown in Scheme 3, the carboxylic acid of a MEB-containing dipeptide is coupled to the free amino group of a peptide linked to a solid support. The MEB-containing dipeptide can include an amino protecting group, such as Fmoc (as shown in Scheme 3) or Boc. The MEB-containing dipeptide can be deprotected to remove the amino protecting group, and a subsequent amino acid added to the peptide.

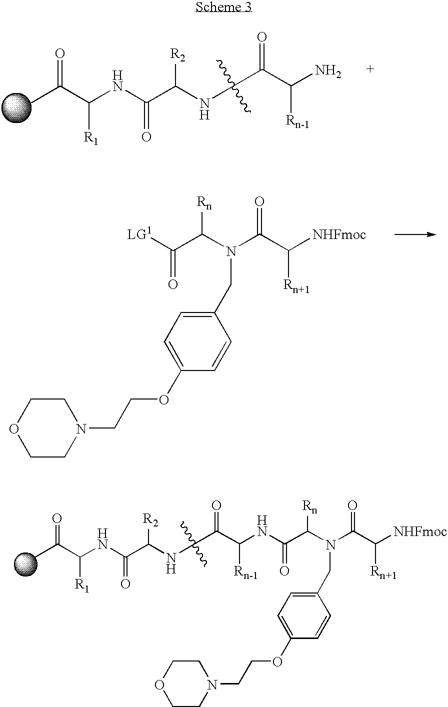

In some circumstances, it can be desirable to prepare a long peptide (e.g., a peptide of more than 40, more than 50, more than 75, more than 100, or 150 or more residues) by first synthesizing two (or more) shorter peptides. The shorter peptides can be joined using native chemical ligation to afford the longer peptide (see, for example, Dawson, P. E. et al., Science (1994) 266, 776, which is incorporated by reference in its entirety). In general, native chemical ligation allows the formation of a longer peptide from two shorter peptides, one having a C-terminal thioester (e.g., an aryl thioester), and the other peptide having an N-terminal cysteine residue. In order to perform native chemical ligation, both peptides should be water-soluble and highly pure. If one of the shorter peptides includes a difficult sequence, it can be prepared with a backbone nitrogen modifying group. The native chemical ligation can be performed prior to removal of the backbone nitrogen modifying group. The ligated peptide can assume its proper 3-dimensional fold after removal of the backbone nitrogen modifying group.

### EXAMPLES

### Synthesis of MEBA

4-(2-morpholinoethoxy)benzonitrile: 6.1 g (0.051 mol) of 4-cyanophenol (Aldrich) was combined with 14.3 g (0.0768 mol) of 4-(2-chloroethyl)morpholine hydrochloride (Aldrich), 21.1 g (0.153 mol) of potassium carbonate and 2 g of potassium iodide in 250 mL of anhydrous dimethylformamide (DMF) and heated at 60° C. for 4 hours. The reaction was monitored by LC/MS and TLC (EtOAc/hexane 1:1 or 100% EtOAc). The TLC plate was deactivated with 1% triethylarmnie (TEA) in hexane. After the completion of the reaction the reaction mixture was filtered and the precipitate washed three times with 10 mL of DMF. The filtrate was concentrated by rotary evaporator. The residue was dissolved in dichloromethane and filtered through silica pretreated with 1% TEA in 10% EtOAc/hexane, and rinsed with ethyl acetate. Yield: 9.6 g (81%) of 4-(2-morpholinoethoxy)benzonitrile compound as white to slightly pink needles.

4-(2-morpholinoethoxy)benzylamine (MEBA): 10 mL of a 1.0 M solution of LiAlH₄ in tetrahydrofuran (THF) was added dropwise to a stirred, cooled (0-4° C.) solution of 1.15 g of 4-(2-morpholinoethoxy)benzonitrile in anhydrous THF. After the addition was completed, the reaction was allowed to warm to room temperature. The reaction was monitored by LC/MS for disappearance of the starting material and also for the aldehyde side product (M+H=236). The presence of the aldehyde side product can indicate an incomplete reduction, as the aldehyde forms from unreacted imine during analysis. After 6-8 hours, the reaction mixture was added slowly to a stirred, cooled solution of saturated NH₄Cl (50 mL), stirred for 30 minutes, and filtered. The filtrate was extracted with a 25 mL portion of ethyl acetate. The aqueous phase was separated and a portion of 50% NaOH half the volume of the aqueous phase was added. This mixture was extracted twice with 50 mL of dioxane. Combined dioxane extracts were stirred for 30 minutes with solid potassium hydroxide (˜1 g) to remove water. The potassium hydroxide slurry was removed and the solvent removed by rotary evaporator. The resulting colorless oil crystallized when allowed to stand at 4° C. Yield: 980 mg, 84%.

Introduction of a MEB Group into a Peptide

Method 1. The MEB group was added to a pre-selected sites (e.g., at a glycine residue) of a peptide sequence using alkylation with a resin-bound alpha-bromo carboxamide. Bromoacetic anhydride was freshly prepared from bromoacetic acid (3.2 mmol, 444.64 mg). The bromoacetic anhydride and diisopropylcarbodiimide (0.16 mmol, 0.025 mL) in chilled dichloromethane (12 mL) was added to the N-terminus of a resin-supported growing peptide chain (0.16 mmol, 400 mg). The peptide was synthesized using standard Fmoc protocols. The mixture was gently shaken at 20° C. for 1 hour. The mixture was filtered using a 50 mL polypropylene filtration tube. The resin was washed with dichloromethane (4×10 mL). MEBA (5.25 mmol, 1.24 g) in dichloromethane (2 mL) was added to the resin. The mixture was gently shaken for 20 hours at 20° C. The resin was washed with dichloromethane (4×5 mL). Using method 1, the carbonyl carbon and alpha carbon of the protected residue (glycine) are derived from bromoacetic anhydride, and the backbone nitrogen of the protected residue is derived from MEBA. The synthesis of the peptide chain was then continued using standard Fmoc protocols. Multiple MEB groups can be inserted into a peptide chain using the method described above at multiple sites along the chain.

Method 2. In this method the pre-selected sites of the peptide sequence were modified with a dipeptide unit (see below) in which the amide bond has been protected with a MEB group. The protected dipeptide unit was then coupled to the N-terminus of a growing peptide and the synthesis was continued using standard Fmoc protocols.

Preparation of a MEB-protected dipeptide. To a bromoacetic acid pre-loaded HMP-resin (1 mmol, 2.5 g), MEBA (5.2 mmol, 1.23 g) in dichloromethane (10 mL) was added and the mixture was shaken at 20° C. for 20 hours. The mixture was filtered using a 50 mL polypropylene filtration tube. The resin was washed with dichloromethane (4×10 mL). A mixture of N-alpha-Fmoc-protected amino acid (5 mmol) in N,N-dimethylformamide (20 mL), bromotripyrrolidinophosphonium hexafluorophosphate (4.8 mmol, 2.24 g) and N,N-diisopropylethylamine (10 mmol, 1.74 mL) were mixed for 5 minutes at 20° C. before adding to the resin. The resin was shaken at 20° C. for 18 hours. The resin was then washed with N,N-dimethylformamide (4×10 mL) and dichloromethane (2×10 mL). The resin was dried in vacuo. The dipeptide was cleaved from the resin with trifluoroacetic acid/water, 9/1 (10.0 mL) at 20° C. for 2 hours. The trifluoroacetic acid-resin mixture was filtered to remove the resin. Trifluoroacetic acid was removed under reduced pressure to give the MEB-protected dipeptide unit. The dipeptide can then be used in SPPS of a longer peptide.

Removal of MEB Group from a Synthetic Peptide

A 50-mL Teflon tube containing a mixture of MEBA-modified peptide (0.018 mmol, 25 mg) and p-cresol (400 mg) was mounted onto an HF apparatus. The tube was immersed into a dry ice acetone bath and anhydrous hydrogen fluoride (10 mL) was condensed. Next, the dry ice acetone bath was replaced by a water bath containing crushed ice, and the reaction was magnetically stirred for 1 hour. The hydrogen fluoride was evaporated from the Teflon tube and trapped into a 15% solution of potassium hydroxide using nitrogen gas at 20 psi for 30 minutes. The Teflon tube was removed from the HF apparatus and the peptide was precipitated with chilled ethyl ether. The solid peptide was then taken up with a 50% solution of acetonitrile-water. The solution was frozen and lyophilized to give the MEB-deprotected peptide.

Removal of Backbone Nitrogen Modifying Groups

Table 1 summarizes the results of experiments testing the removal of backbone nitrogen modifying groups under various conditions.

TABLE 1

| modifying group | | removal conditions | % cleavage |
|---|---|---|---|
| benzyl | 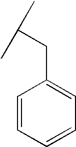 | HF, 1 hour, 0° C.TFA, 3 hoursRaney nickel, 6 hours, roomtemperature, pH 6.0 | 0 0 0 |
| p-methylbenzyl | 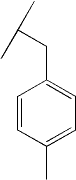 | HF, 1 hour, 0° C.TFA, 3 hours | 50 0 |
| p-methoxybenzyl | 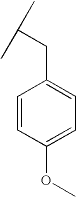 | HF, 1 hour, 0° C.TFA, 3 hoursRaney nickel, 6 hours, roomtemperature, pH 6.0 | 100 0 0 |
| N-p-acetylbenzyl | 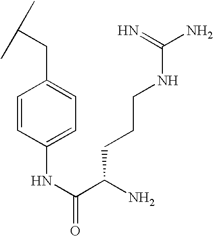 | HF, 1 hour, 0° C.H₂, 10% Pd/C | 0 0 |
| MEB | 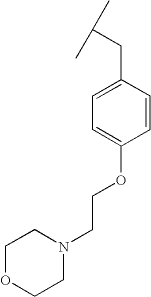 | HF, 1 hour, 0° C.TFA, 3 hours | 100 0 |

Synthesis of Polypeptide

To test the ability of MEB to prevent peptide aggregation during solid phase peptide synthesis, a peptide fragment with high content of beta sheet secondary structure was prepared. The sequence:

CEAKPWYEPIYLGGVFQLEKGDRLSAEINRPDYLLFAESGQVYFGIIAL
corresponds to the C-terminus of human TNF-alpha. The 48-amino acid peptide was prepared by solid phase peptide synthesis using Fmoc-protected amino acids on an Applied Biosystems 433A peptide synthesizer according to manufacture specific protocols. A commercial available Fmoc-Leu-Wang Resin (0.47 g. 0.42 mmol/g) was loaded into the reaction vessel and the sequence was extended using five-fold excess of activated Fmoc-amino acids during every coupling step. Fmoc-amino acids were activated by the addition of equimolar amounts of HBTU and HOBt and 2 equivalents of DIEA in DMF. Three MEB groups were introduced at 013, G40 and G45 using method 1. The peptide was cleaved from the resin and deprotected with TFA/EDT/TA/phenol/water/TIPS (68.5:10:10:5:3.5:1 V:V). The TFA resin mixture was filtered. Chilled diethyl ether was added to the filtrate to precipitate the peptide, which was then centrifuged at 2500 RPM for 5 minutes. The pellet was washed three more times with chilled diethyl ether. The peptide was subsequently dissolved in 95% acetic acid and lyophilized. The peptide was purified by reverse-phase HPLC (>98% purity) on a Varian 210 HPLC system with 214-nm UV detection, using a Higgins Analytical C8 column (2×25 cm), a linear gradient of 25-65% acetonitrile over 45 min, and a flow rate of 5 mL/min in 0.1% CF₃CO₂H. The LC-ESI-MS purified peptide showed the correct mass (M+1 6,239.24).

Attempts to make the same 48 amino acid peptide using traditional solid phase peptide synthesis without introducing the three MEB groups at G13, G40 and G45 were unsuccessful.

Other embodiments are within the scope of the following claims.

What is claimed is:

1. A method of making a peptide comprising forming a peptide including a backbone nitrogen modifying group, wherein the backbone nitrogen modifying group includes a substituted aryl group, the substituted aryl group including a directing moiety and a hydrophilic moiety.

2. The method of claim 1, wherein the peptide is linked to a solid support.

3. The method of claim 1, wherein the peptide includes at least one commonly occurring natural amino acid residue, wherein the commonly occurring natural amino acid optionally includes a protecting group.

4. The method of claim 1, wherein the peptide includes at least one non-naturally occurring amino acid residue.

5. The method of claim 1, further comprising adding an amino acid residue to the peptide, thereby extending the peptide.

6. The method of claim 2, further comprising cleaving the peptide from the solid support, wherein cleaving the peptide does not substantially remove the backbone nitrogen modifying group from the peptide.

7. The method of claim 1, further comprising removing the backbone nitrogen modifying group from the peptide.

8. The method of claim 1, wherein the substituted aryl group is a substituted phenyl group.

9. The method of claim 8, wherein the substituted phenyl group is ortho-unsubstituted.

10. The method of claim 1, wherein the hydrophilic moiety includes a tertiary amine.

11. The method of claim 1, wherein the peptide is substantially water-soluble.

12. The method of claim 1, wherein the peptide has the formula: 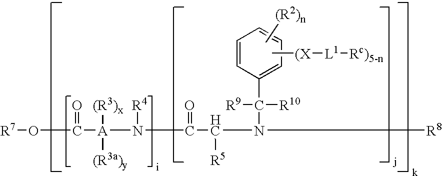
wherein:
X is O, S, NH, or a bond;
each L¹, independently, is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—;
each R^{c}, independently, is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo, wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R⁹, independently, is hydrogen, alkyl, aryl, substituted alkyl, substituted aryl, or halo;
each R¹⁰, independently, is hydrogen, alkyl, aryl, substituted alkyl, substituted aryl, or halo;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
each R⁵, independently, is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R⁷ is hydrogen, alkyl, aryl, aralkyl, or a solid support;
R⁸ is hydrogen, alkyl, an amino protecting group, or has the formula —C(═O)CH(R⁵)R⁶, wherein R⁶ is a leaving group;
each i, independently, is zero or a positive integer;
each j, independently, is zero or a positive integer;
k is a positive integer;
m is 1 or 2;
n is 0, 1, 2, 3 or 4;
each x, independently, is 1, 2, 3, 4, or 5;
each y, independently, is 1, 2, 3, 4, or 5;
z is 1, 2, 3, 4, 5 or 6.

13. The method of claim 12, wherein the peptide includes at least one -L¹-R^{c}.

14. The method of claim 12, wherein the peptide has the formula: 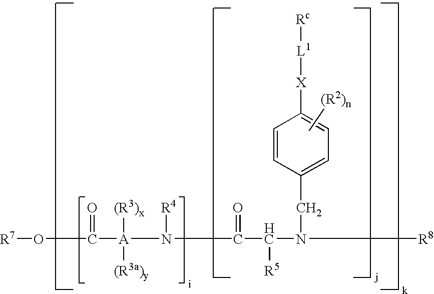
wherein:
X is O, S, NH, or a bond;
each L¹, independently, is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—;
each R^{c}, independently, is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo, wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{G}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
each R⁵, independently, is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R⁷ is hydrogen, alkyl, aryl, aralkyl, or a solid support;
R⁸ is hydrogen, alkyl, an amino protecting group, or has the formula —C(═O)CH(R⁵)R⁶, wherein R⁶ is a leaving group;
each i, independently, is zero or a positive integer;
each j, independently, is zero or a positive integer;
k is a positive integer;
m is 1 or 2;
n is 0, 1, 2, 3 or 4;
each x, independently, is 1, 2, 3, 4, or 5;
each y, independently, is 1, 2, 3, 4, or 5;
z is 1, 2, 3, 4, 5 or 6.

15. The method of claim 14, wherein X is O.

16. The method of claim 15, wherein L¹ is C₁-C₄ alkylene.

17. The method of claim 16, wherein R^{c} is heterocycloalkyl.

18. The method of claim 14, wherein —X-L¹-R^{c} is 2-(morpholin-4-yl)ethoxy.

19. The method of claim 12, wherein each R⁵, independently, is hydrogen or alkyl.

20. The method of claim 12, wherein R⁷ is a solid support and R⁸ is an amino protecting group.

21. The method of claim 12, wherein each A is C₁ alkylene and each R^{3a} is hydrogen.

22. The method of claim 12, wherein the total of all i and all j is less than 300.

23. The method of claim 12, wherein the peptide has a molecular weight of no greater than 40 kDa.

24. The method of claim 12, wherein R⁸ has the formula —C(═O)CH(R⁵)R⁶, wherein R⁶ is a leaving group.

25. The method of claim 24, wherein each j is zero.

26. The method of claim 24, wherein R⁶ is halo.

27. The method of claim 24, further comprising contacting the peptide with a compound having the formula: 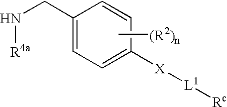
wherein:
X is O, S, NH, or a bond;
L¹ is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S, S(O)ₘ—, —NR^{d}SO₂—, —SO₂NR^{d}—, or —NR^{d}—;
R^{c} is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
R^{4a} is hydrogen, alkyl, or an amino protecting group;
m is 1 or 2;
n is 0, 1, 2, 3, or 4; and
z is 1, 2, 3, 4, 5, or 6.

28. The method of claim 27, wherein the compound is 4-(2-(morpholin-4-yl)ethoxy)benzylamine.

29. The method of claim 28, further comprising adding an amino acid residue to the peptide, thereby forming a longer peptide.

30. The method of claim 12, further comprising contacting the peptide with a compound having the formula: 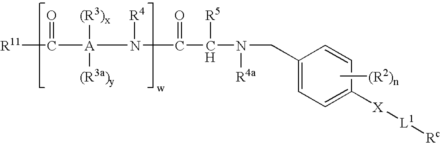
wherein:
X is O, S, NH, or a bond;
L¹ is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—;
R^{c} is —NR^{a}R^{b}, —OR^{a}, —SR^{a}, S(O)ₘR^{a}, —S(O)₂NR^{a}R^{b}, S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo wherein R² is optionally substituted with -L₁-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
R^{4a} is hydrogen, alkyl, or an amino protecting group;
R⁵ is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R¹¹ is hydroxy, alkoxy, aryloxy, aralkyloxy, or a leaving group;
m is 1 or 2;
n is 0, 1, 2, 3, or 4;
w is 0, 1, or 2;
each x, independently, is 1, 2, 3, 4, or 5;
each y, independently, is 1, 2, 3, 4, or 5; and
z is 1, 2, 3, 4, 5, or 6.

31. The method of claim 12, further comprising contacting the peptide with a compound having the formula: 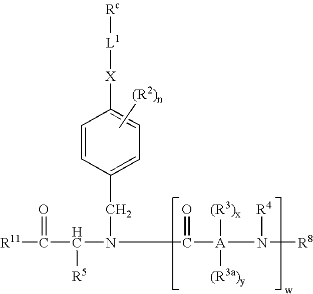
wherein:
X is O, S, NH, or a bond;
L¹ is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—;
R^{c} is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, −O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, NR^{a}R^{b}, —NR^{a}C(═O)R^{b}, or halo wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
R⁵ is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R¹¹ is hydroxy, alkoxy, aryloxy, aralkyloxy, or a leaving group;
m is 1 or 2;
n is 0, 1, 2, 3, or 4;
w is 0, 1, or 2;
each x, independently, is 1, 2, 3, 4, or 5;
each y, independently, is 1, 2, 3, 4, or 5; and
z is 1, 2, 3, 4, 5, or 6.

32. A composition comprising a peptide including a backbone nitrogen modifying group, wherein the backbone nitrogen modifying group includes a substituted aryl group, the substituted aryl group including a directing moiety and a hydrophilic moiety.

33. The composition of claim 32, wherein the peptide includes a plurality of backbone nitrogen modifying groups.

34. The composition of claim 32, wherein the peptide is linked to a solid support.

35. The composition of claim 32, wherein the peptide includes at least one commonly occurring natural amino acid residue, wherein the commonly occurring natural amino acid optionally includes a protecting group.

36. The composition of claim 32, wherein the peptide includes at least one non-naturally occurring amino acid residue.

37. The composition of claim 32, wherein the substituted aryl group is a substituted phenyl group.

38. The composition of claim 32, wherein the substituted phenyl group is ortho-unsubstituted.

39. The composition of claim 32, wherein the hydrophilic moiety includes a tertiary amine.

40. The composition of claim 32, wherein the peptide is substantially water-soluble.

41. The composition of claim 32, wherein the peptide has the formula: 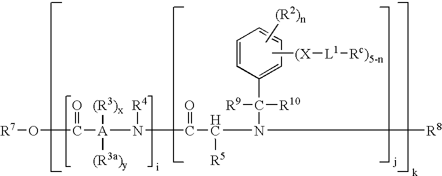
wherein:
X is O, S, NH, or a bond;
each L¹, independently, is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, SO₂NR^{d}—, or —NR^{d}—;
each R^{c}, independently, is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R⁹, independently, is hydrogen, alkyl, aryl, substituted alkyl, substituted aryl, or halo;
each R¹⁰, independently, is hydrogen, alkyl, aryl, substituted alkyl, substituted aryl, or halo;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
each R⁵, independently, is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g} —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R⁷ is hydrogen, alkyl, aryl, aralkyl, or a solid support;
R⁸ is hydrogen, alkyl, an amino protecting group, or has the formula —C(═O)CH(R⁵)R⁶, wherein R⁶ is a leaving group;
each i, independently, is zero or a positive integer;
each j, independently, is zero or a positive integer, provided that at least one j is a positive integer;
k is a positive integer;
m is 1 or 2;
n is 0, 1, 2, 3 or 4; and
each x, independently, is 1, 2, 3, 4, or 5;
each y, independently, is 1, 2, 3, 4, or 5; and
z is 1, 2, 3, 4, 5 or 6.

42. The composition of claim 41, wherein the peptide includes at least one -L¹-R^{c}.

43. The composition of claim 41, wherein the peptide has the formula: 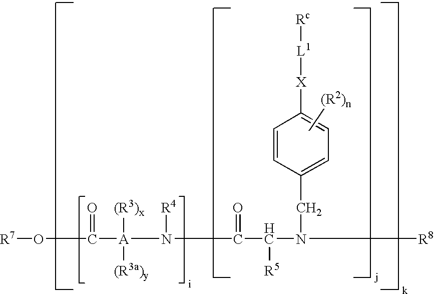
wherein:
X is O, S, NH, or a bond;
each L¹, independently, is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, S(O)ₘ—, —NR^{d}SO₂—, —SO₂NR^{d}—, or —NR^{d}—;
each R^{c}, independently, is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo, wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
each R⁵, independently, is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R⁷ is hydrogen, alkyl, aryl, aralkyl, or a solid support;
R⁸ is hydrogen, alkyl, an amino protecting group, or has the formula —C(═O)CH(R⁵)R⁶, wherein R⁶ is a leaving group;
each i, independently, is zero or a positive integer;
each j, independently, is zero or a positive integer;
k is a positive integer;
m is 1 or 2;
n is 0, 1, 2, 3 or 4;
each x, independently, is 1, 2, 3, 4, or 5;
each y, independently, is 1, 2, 3, 4, or 5;
z is 1, 2, 3, 4, 5 or 6.

44. The composition of claim 43, wherein X is O.

45. The composition of claim 44, wherein L¹ is C₁-C₄ alkylene.

46. The composition of claim 44, wherein R^{c} is heterocycloalkyl.

47. The composition of claim 43, wherein —X-L¹-R^{c} is 2-(morpholin-4-yl)ethoxy.

48. The composition of claim 41, wherein each R⁵, independently, is hydrogen or alkyl.

49. The composition of claim 41, wherein R⁷ is a solid support and R⁸ is an amino protecting group.

50. The composition of claim 41, wherein R⁷ is hydrogen and R⁸ is hydrogen.

51. The composition of claim 41, wherein each A is C₁ alkylene and each R^{3a} is hydrogen.

52. The composition of claim 41, wherein the total of all i and all j is less than 300.

53. The composition of claim 41, wherein the peptide has a molecular weight of no greater than 40 kDa.

54. The composition of claim 41, wherein R⁸ has the formula —C(═O)CH(R⁵)R⁶, wherein R⁶ is a leaving group.

55. The composition of claim 54, wherein R⁶ is halo.

56. A compound having the formula: 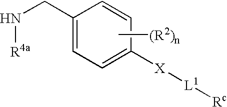
wherein:
X is O, S, NH, or a bond;
L¹ is C₁-C₁₀ alkylene, alkenylene, alkynylene, cycloalkylene, arylene, or aralkylene, wherein L¹ is optionally interrupted by one or more of —C(O)—, —O—, —C(O)NR^{d}, —NR^{d}—C(O)—, —NR^{d}C(O)NR^{d}—, —OC(O)NR^{d}—, —NR^{d}—C(O)—, —S—, S(O)ₘ—, —NR^{d}SO₂—, —SO₂NR^{d}—, or —NR^{d}—;
R^{c} is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, SR^{a}, —NR^{a}R^{b}, NR^{a}C(═O)R^{b}, or halo wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
R^{4a} is hydrogen, alkyl, or an amino protecting group;
m is 1 or 2;
n is 0, 1, 2, 3, or 4; and
z is 1, 2, 3, 4, 5, or 6.

57. A compound having the formula: 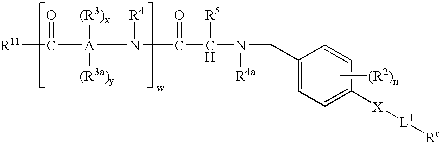
wherein:
X is O, S, NH, or a bond;
L¹ is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—;
R^{c} is —NR^{a}R^{b}, —OR^{a}, —SR^{a}, —S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
R^{4a} is hydrogen, alkyl, or an amino protecting group;
R⁵ is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R¹¹ is hydroxy, alkoxy, aryloxy, aralkyloxy, a solid support, or a leaving group;
m is 1 or 2;
n is 0, 1, 2, 3, or 4;
w is 0, 1, or 2;
each x, independently, is 1, 2, 3, 4, or 5;
each y, independently, is 1, 2, 3, 4, or 5; and
z is 1, 2, 3, 4, 5, or 6.

58. A compound having the formula: 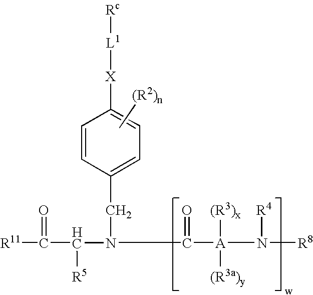
wherein:
X is O, S, NH, or a bond;
L¹ is C₁-C₁₀ alkylene, alkenylene, alkynylene, cycloalkylene, arylene, or aralkylene, wherein L¹ is optionally interrupted by one or more of —C(O)—, —O—, —C(O)NR^{d}—, —NR^{d}—C(O)—, —NR^{d}C(O)NR^{d}—, —OC(O)NR^{d}—, —NR^{d}—C(O)—O—, —S—, —S(O)ₘ—, —NR^{d}SO₂—, —SO₂NR^{d}—, or —NR^{d}—;
R^{c} is —NR^{a}R^{b}, —OR^{a}, —SR^{a}, S(O)ₘR^{a}, —S(O)₂NR^{a}R^{b}, —S(O)ₘOR^{a}, —NR^{d}C(O)R^{e}, —O(CR^{d}R^{e})_{z}NR^{a}R^{b}, —C(O)R^{a}, —C(O)NR^{d}R^{e}, —NR^{a}C(O)R^{b}, —OC(O)NR^{a}R^{b}, —NR^{d}C(O)OR^{a}, —NR^{d}C(O)NR^{a}R^{b}, heterocycloalkyl, or (heterocycloalkyl)alkyl;
each R², independently, is hydrogen, —R^{a}, —OR^{a}, —SR^{a}, —NR^{a}R^{b}, —NR^{a}C(═O)R^{b}, or halo wherein R² is optionally substituted with -L¹-R^{c};
each R^{a}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{b}, independently, is hydrogen, alkyl, alkenyl, alkynyl, cycloalkyl, (cycloalkyl)alkyl, cycloalkenyl, heterocycloalkyl, (heterocycloalkyl)alkyl, aryl, aryl-fused cycloalkyl, aralkyl, aryl-substituted alkenyl, aryl-substituted alkynyl, cycloalkenyl-substituted cycloalkyl, or biaryl;
each R^{d}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each R^{e}, independently, is hydrogen, alkyl, alkenyl, alkynyl, cycloalkyl, (cycloalkyl)alkyl, cycloalkenyl, heterocycloalkyl, (heterocycloalkyl)alkyl, or aryl;
each A, independently, is C₁-C₁₀ alkylene, alkenylene, alkynylene, cycloalkylene, arylene, or aralkylene, wherein A optionally includes 1-3 heteroatoms selected from N, O and S;
each R³, independently, is hydrogen, alkyl, alkenyl, alkynyl, cycloalkyl, heterocycloalkyl, aralkyl, (cycloalkyl)alkyl, (heterocycloalkyl)alkyl or aryl; R³ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(NH)NR^{f}R^{g};
each R^{3a}, independently, is hydrogen, alkyl, alkenyl, alkynyl, cycloalkyl, heterocycloalkyl, aralkyl, (cycloalkyl)alkyl, (heterocycloalkyl)alkyl or aryl; R^{3a} being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g}; or R³ and R^{3a} together with the atoms to which they are attached form a 3-14 membered cyclic, bicyclic or tricyclic moiety, optionally including 1-6 heteroatoms selected from N, O, and S;
each R⁴, independently, is hydrogen or alkyl; or R³ and R⁴ together with the atoms to which they are attached form a 3-14 membered cyclic, bicyclic or tricyclic moiety, optionally including 1-6 heteroatoms selected from N, O, and S;
R⁵ is hydrogen, alkyl, alkenyl, alkynyl, cycloalkyl, heterocycloalkyl, aralkyl, (cycloalkyl)alkyl, (heterocycloalkyl)alkyl or aryl; R⁵ being optionally substituted with —OR^{f}, —SR^{f}, —CO₂R^{f}, halo, haloalkyl, —CN, —NO₂, —NR^{f}R^{g}, —C(═O)NR^{f}R^{g}, or —NHC(═NH)NR^{f}R^{g};
each R^{f}, independently, is hydrogen, alkyl, aryl, aralkyl, or a protecting group;
each R^{g}, independently, is hydrogen, alkyl, aryl, aralkyl, or a protecting group;
R¹¹ is hydroxy, alkoxy, aryloxy, aralkyloxy, a solid support, or a leaving group;
m is 1 or 2;
n is 0, 1, 2, 3, or 4;
w is 0, 1, or 2;
each x, independently, is 1, 2, 3, 4, or 5;
each y, independently, is 1, 2, 3, 4, or 5; and
z is 1, 2, 3, 4, 5, or 6.

59. A method of making a peptide having a predetermined amino acid sequence comprising:
determining a beta-sheet-forming propensity for at least a portion of the amino acid sequence; and
selecting an amino acid residue of the sequence for modification with a backbone nitrogen modifying group based on the determined beta-sheet-forming propensity.

60. The method of claim 59, wherein the backbone nitrogen modifying group includes a substituted aryl group, the substituted aryl group including a directing moiety and a hydrophilic moiety.
